(19) United States
(12) Patent Application Publication
(10) Pub. No.: US 2014/0155347 A1
(43) Pub. Date: Jun. 5, 2014

(54) Spray Drying of High Molecular Weight Hyaluronic Acid

(71) Applicant: NOVOZYMES BIOPHARMA DK A/S, Bagsvaerd (DK)

(72) Inventors: Kristoffer Toemmeraas, Bunkeflostrand (SE); Poul Bach, Birkeroed (DK)

(21) Appl. No.: 14/119,099

(22) PCT Filed: May 29, 2012
(86) PCT No.: PCT/EP2012/059959

§ 371 Date: Feb. 14, 2014

(30) Foreign Application Priority Data
May 30, 2011 (EP) 11168106.0

(51) Int. Cl. A61K 31/728; C08B 37/08

(52) U.S. Cl. CPC A61K 31/728; C08B 37/0072
USPC 514/54; 536/53

(57) ABSTRACT

A method of producing spray-dried hyaluronic acid comprising a) spray drying hyaluronic acid, wherein the concentration of the hyaluronic acid in the feed to the spray dryer is in the range of from 3.5 g/l to 7.0 g/l; b) having the temperature in the feed to the spray dryer in the range of from 0° C. to 100° C.; and wherein the molecular weight of the hyaluronic acid in the feed to the spray dryer is ≧1200 kDa.

### FIELD OF INVENTION

The present invention relates to spray drying of polysaccharides, in particular hyaluronic acid or a salt thereof.

### BACKGROUND

Hyaluronic acid (HA) is a natural and linear carbohydrate polymer belonging to the class of non-sulfated glycosaminoglycans. It is composed of beta-1,3-N-acetyl glucosamine and beta-1,4-glucuronic acid repeating disaccharide units with a molecular weight (MW) up to 10 MDa. HA is present in hyaline cartilage, synovial joint fluid, and skin tissue, both dermis and epidermis.

HA may be extracted from natural tissues including the connective tissue of vertebrates, from the human umbilical cord and from cocks' combs. However, it is preferred today to prepare it by microbiological methods to minimize the potential risk of transferring infectious agents, and to increase product uniformity, quality and availability (U.S. Pat. No. 6,951,743).

Numerous roles of HA in the body have been identified. It plays an important role in biological organisms, as a mechanical support for cells of many tissues, such as skin, tendons, muscles and cartilage. HA is involved in key biological processes, such as the moistening of tissues, and lubrication. It is also suspected of having a role in numerous physiological functions, such as adhesion, cell motility, cancer, angiogenesis, and wound healing.

Due to the unique physical and biological properties of HA (including visco-elasticity, biocompatibility, and biodegradability), HA is employed in a wide range of current and developing applications within cosmetics, ophthalmology, rheumatology, drug and gene delivery, wound healing and tissue engineering.

WO 05/116531 describes a process of spray drying hyaluronic acid (with a molecular weight of 800 kDa—see Example 6).

A significant loss in molecular weight may be seen when spray drying hyaluronic acid with a higher molecular weight than around 1200 kDa.

### SUMMARY OF THE INVENTION

The present invention relates to a method of spray drying hyaluronic acid with a high molecular weight. The method comprises
a) spray drying hyaluronic acid, wherein the concentration of the hyaluronic acid in the feed to the spray dryer is in the range of from 3.5 g/l to 7.0 g/l;
b) having the temperature in the feed to the spray dryer in the range of from 0° C. to 100° C.; and
wherein the molecular weight of the hyaluronic acid in the feed to the spray dryer is ≧1200 kDa.

The process of the present invention reduces the loss of the molecular weight of the spray dried hyaluronic acid product.

### DETAILED DESCRIPTION

The present invention relates to a method of spray drying hyaluronic acid with a high molecular weight.

### Hyaluronic Acid

“Hyaluronic acid” is a polysaccharide defined herein as an unsulphated glycosaminoglycan composed of repeating disaccharide units of N-acetylglucosamine (GlcNAc) and glucuronic acid (GlcUA) linked together by alternating beta-1,4 and beta-1,3 glycosidic bonds. Hyaluronic acid is also known as hyaluronan, hyaluronate, or HA. The terms hyaluronan and hyaluronic acid are used interchangeably herein.

Rooster combs are a significant commercial source for hyaluronan. Microorganisms are an alternative source. U.S. Pat. No. 4,801,539 discloses a fermentation method for preparing hyaluronic acid involving a strain of Streptococcus zooepidemicus. WO 03/054163 discloses a fermentation method for preparing hyaluronic acid involving a Bacillus host.

Hyaluronan synthases have been described from vertebrates, bacterial pathogens, and algal viruses (DeAngelis, P. L., 1999, Cell. Mol. Life. Sci. 56: 670-682). WO 99/23227 discloses a Group I hyaluronate synthase from Streptococcus equisimilis. WO 99/51265 and WO 00/27437 describe a Group II hyaluronate synthase from Pasturella multocida. Ferretti et al. disclose the hyaluronan synthase operon of Streptococcus pyogenes, which is composed of three genes, hasA, hasB, and hasC, that encode hyaluronate synthase, UDP glucose dehydrogenase, and UDP-glucose pyrophosphorylase, respectively (Proc. Natl. Acad. Sci. USA. 98, 4658-4663, 2001). WO 99/51265 describes a nucleic acid segment having a coding region for a Streptococcus equisimilis hyaluronan synthase.

Since the hyaluronan of a recombinant Bacillus cell is expressed directly to the culture medium, a simple process may be used to isolate the hyaluronan from the culture medium. First, the Bacillus cells and cellular debris are physically removed from the culture medium. The culture medium may be diluted first, if desired, to reduce the viscosity of the medium. Many methods are known to those skilled in the art for removing cells from the culture medium, such as centrifugation or microfiltration. The remaining supernatant may then be filtered, such as by ultrafiltration, to concentrate and remove small molecule contaminants from the hyaluronan. Following removal of the cells and cellular debris, a simple precipitation of the hyaluronan from the medium may be performed by known mechanisms. Salt, alcohol, or combinations of salt and alcohol may be used to precipitate the hyaluronan from the filtrate.

The hyaluronan may be dried from any solution, e.g., from a filtrate or from a re-dissolved solution, by using, e.g., the spray drying method according to the present invention.

### Host Cells

A preferred embodiment relates to the product of the first aspect, wherein the hyaluronic acid or salt thereof is recombinantly produced, preferably by a Gram-positive bacterium or host cell, more preferably by a bacterium of the genus Bacillus.

The host cell may be any Bacillus cell suitable for recombinant production of hyaluronic acid. The Bacillus host cell may be a wild-type Bacillus cell or a mutant thereof. Bacillus cells useful in the practice of the present invention include, but are not limited to, Bacillus agaraderhens, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells. Mutant Bacillus subtilis cells particularly adapted for recombinant expression are described in WO 98/22598. Non-encapsulating Bacillus cells are particularly useful in the present invention.

In a preferred embodiment, the Bacillus host cell is a Bacillus amyloliquefaciens, Bacillus clausii, Bacillus lentus, Bacillus licheniformis, Bacillus stearothermophilus or Bacillus subtilis cell.

### Production

In the method of the present invention, the cells (e.g., Streptococcus) or the host cells (e.g., Bacillus) are cultivated in a nutrient medium suitable for production of the hyaluronic acid using methods known in the art.

For example, the cells may be cultivated by shake flask cultivation, small-scale or large-scale fermentation (including continuous, batch, fed-batch, or solid state fermentations) in laboratory or industrial fermentors.

The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection).

The level of hyaluronic acid may be determined as known in the art, e.g., by using the modified carbazole method (Bitter and Muir, 1962, Anal Biochem. 4: 330-334).

### Molecular Weight

The average molecular weight of the hyaluronic acid may be measured as known in the art.

In particular, the average molecular weight of the hyaluronic acid may be determined using standard methods in the art, such as those described by Ueno et al., 1988, Chem. Pharm. Bull. 36, 4971-4975; Wyatt, 1993, Anal. Chim. Acta 272: 1-40; and Wyatt Technologies, 1999, “Light Scattering University DAWN Course Manual” and “DAWN EOS Manual” Wyatt Technology Corporation, Santa Barbara, Calif.

Molecular weight determination of hyaluronic acid may also be performed using GPC-RI-LS wherein the molecular weight determination of hyaluronic acid is performed using GPC coupled with differential RI and multi-angle light-scattering detectors.

In a preferred embodiment, the molecular weight of the hyaluronic acid in the feed to the spray dryer is at least 1200 kDa; in particular the molecular weight of the hyaluronic acid in the feed to the spray dryer is in the range of from 1200 kDa to 10,000 kDa; preferably the molecular weight of the hyaluronic acid in the feed to the spray dryer is in the range of from 1200 kDa to 9,500 kDa; more preferably the molecular weight of the hyaluronic acid in the feed to the spray dryer is in the range of from 1200 kDa to 9,000 kDa; more preferably the molecular weight of the hyaluronic acid in the feed to the spray dryer is in the range of from 1200 kDa to 8,500 kDa; more preferably the molecular weight of the hyaluronic acid in the feed to the spray dryer is in the range of from 1200 kDa to 8,000 kDa; more preferably the molecular weight of the hyaluronic acid in the feed to the spray dryer is in the range of from 1200 kDa to 7,500 kDa; more preferably the molecular weight of the hyaluronic acid in the feed to the spray dryer is in the range of from 1200 kDa to 7,000 kDa; more preferably the molecular weight of the hyaluronic acid in the feed to the spray dryer is in the range of from 1200 kDa to 6,500 kDa; more preferably the molecular weight of the hyaluronic acid in the feed to the spray dryer is in the range of from 1200 kDa to 6,000 kDa; more preferably the molecular weight of the hyaluronic acid in the feed to the spray dryer is in the range of from 1200 kDa to 5,500 kDa; more preferably the molecular weight of the hyaluronic acid in the feed to the spray dryer is in the range of from 1200 kDa to 5,000 kDa; more preferably the molecular weight of the hyaluronic acid in the feed to the spray dryer is in the range of from 1200 kDa to 4,500 kDa; more preferably the molecular weight of the hyaluronic acid in the feed to the spray dryer is in the range of from 1200 kDa to 4,000 kDa; more preferably the molecular weight of the hyaluronic acid in the feed to the spray dryer is in the range of from 1200 kDa to 3,500 kDa; more preferably the molecular weight of the hyaluronic acid in the feed to the spray dryer is in the range of from 1200 kDa to 3,000 kDa; more preferably the molecular weight of the hyaluronic acid in the feed to the spray dryer is in the range of from 1200 kDa to 2,900 kDa; more preferably the molecular weight of the hyaluronic acid in the feed to the spray dryer is in the range of from 1200 kDa to 2,800 kDa; more preferably the molecular weight of the hyaluronic acid in the feed to the spray dryer is in the range of from 1200 kDa to 2,700 kDa; more preferably the molecular weight of the hyaluronic acid in the feed to the spray dryer is in the range of from 1200 kDa to 2,600 kDa; more preferably the molecular weight of the hyaluronic acid in the feed to the spray dryer is in the range of from 1200 kDa to 2,500 kDa; more preferably the molecular weight of the hyaluronic acid in the feed to the spray dryer is in the range of from 1200 kDa to 2,400 kDa; more preferably the molecular weight of the hyaluronic acid in the feed to the spray dryer is in the range of from 1200 kDa to 2,300 kDa; and in particular the molecular weight of the hyaluronic acid in the feed to the spray dryer is in the range of from 1200 kDa to 2,200 kDa.

In a preferred embodiment, the hyaluronic acid has a molecular weight loss during spray drying of less than 15%; e.g., the hyaluronic acid has a molecular weight loss during spray drying of less than 14%; the hyaluronic acid has a molecular weight loss during spray drying of less than 13%; the hyaluronic acid has a molecular weight loss during spray drying of less than 12%; the hyaluronic acid has a molecular weight loss during spray drying of less than 11%; the hyaluronic acid has a molecular weight loss during spray drying of less than 10%; the hyaluronic acid has a molecular weight loss during spray drying of less than 9%; the hyaluronic acid has a molecular weight loss during spray drying of less than 8%; the hyaluronic acid has a molecular weight loss during spray drying of less than 7%; the hyaluronic acid has a molecular weight loss during spray drying of less than 6%; in particular, the hyaluronic acid has a molecular weight loss during spray drying of less than 5%.

### HA Salts

A preferred embodiment relates to a product which comprises a salt of hyaluronic acid; in particular an inorganic salt of hyaluronic acid; preferably sodium hyaluronate, potassium hyaluronate, ammonium hyaluronate, calcium hyaluronate, magnesium hyaluronate, zinc hyaluronate, or cobalt hyaluronate.

### Derivatized HA

The hyaluronic acid to be spray dried according to the invention may be derivatized or modified as known in the art.

HA may be derivatized or modified in many different ways, e.g., as described in WO 2007/033677 wherein hyaluronic acid (HA) may react with aryl- or alkyl succinic anhydride (ASA) to produce aryl/alkyl succinic anhydride HA derivatives comprising n repeating units and having the general structural formula (I) at pH 8-9:

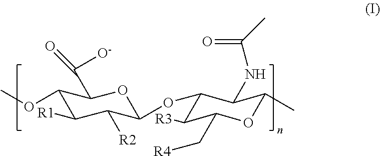

wherein in at least one repeating unit one or more of R1, R2, R3, R4 comprises an esterbound alkyl-/aryl-succinic acid having the general structural formula (II) at pH 8-9, and otherwise R1, R2, R3, R4 are hydroxyl groups, OH:

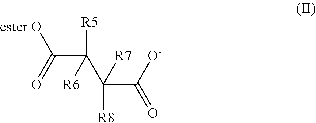

wherein at least one of R5, R6, R7, R8 comprises an alkyl- or aryl-group, and otherwise R5, R6, R7, R8 are hydrogen atoms, H, and wherein the Oxygen labelled “ester” partakes the esterbond with structure (I).

HA may be derivatized or modified as described in WO 2007/106738 wherein an acrylated hyaluronic acid is produced in the following way:

(a) preparing an aqueous liquid with a pH of 7 to 11 comprising hyaluronic acid;
(b) preparing an organic liquid comprising acryl chloride and methylene chloride/diethyl ether; and
(c) mixing the organic liquid of (b) with the aqueous liquid of (a), wherein the pH is maintained between 7 and 11.

The acrylated hyaluronic acid product has the following structure:

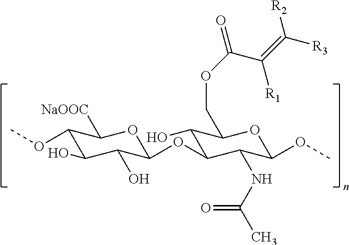

wherein R₁ is selected from the group consisting of hydrogen, methyl, chloride and COCl, and R₂ is selected from the group consisting of hydrogen, methyl, phenyl, chloride, 2-chloro phenyl, COCl and CH₂COCl and R₃ is selected from the group consisting of hydrogen, methyl, chloride, 4-nitro phenyl, 3-trifluoromethylphenyl and styryl moieties.

### The Feed to the Spray Dryer

The concentration of the hyaluronic acid in the feed to the spray dryer should be in the range of from 3.5 g/l to 7.0 g/l; e.g., in the range of from 3.6 g/l to 7.0 g/l; in the range of from 3.7 g/l to 7.0 g/l; in the range of from 3.8 g/l to 7.0 g/l; in the range of from 3.9 g/l to 7.0 g/l; in the range of from 4.0 g/l to 7.0 g/l; in the range of from 4.0 g/l to 6.9 g/l; in the range of from 4.0 g/l to 6.8 g/l; in the range of from 4.0 g/l to 6.7 g/l; in the range of from 4.0 g/l to 6.6 g/l; in the range of from 4.0 g/l to 6.5 g/l; in the range of from 4.0 g/l to 6.4 g/l; in the range of from 4.0 g/l to 6.3 g/l; in the range of from 4.0 g/l to 6.2 g/l; in the range of from 4.0 g/l to 6.1 g/l; and in particular, in the range of from 4.0 g/l to 6.0 g/l.

### Other Ingredients

In one embodiment according to the present invention, the feed comprising the hyaluronic acid may also comprise other ingredients, e.g., an active ingredient and/or an excipient.

Non-limiting examples of an active ingredient or a pharmacologically active substance which may be used in the present invention include protein and/or peptide drugs.

Examples of protein and/or peptide drugs are human growth hormone, bovine growth hormone, porcine growth hormone, growth hormone releasing hormone/peptide, granulocyte-colony stimulating factor, granulocyte macrophage-colony stimulating factor, macrophage-colony stimulating factor, erythropoietin, bone morphogenic protein, interferon or a derivative thereof, insulin or a derivative thereof, atriopeptin-III, monoclonal antibody, tumor necrosis factor, macrophage activating factor, interleukin, tumor degenerating factor, insulin-like growth factor, epidermal growth factor, tissue plasminogen activator, factor IIV, factor IIIV, and urokinase.

An excipient may be included according to the present invention, e.g., for the purpose of stabilizing the active ingredient(s), such excipient may include a protein, e.g., albumin or gelatin; an amino acid, such as glycine, alanine, glutamic acid, arginine, lysine and a salt thereof; a carbohydrate such as glucose, lactose, xylose, galactose, fructose, maltose, saccharose, dextran, mannitol, sorbitol, trehalose and chondroitin sulphate; an inorganic salt such as phosphate; a surfactant such as TWEEN® (ICI), poly ethylene glycol, and a mixture thereof.

### Spray Drying

Spray drying involves the atomization of a liquid feedstock into a spray of droplets and contacting the droplets with hot air in a drying chamber. The sprays are produced by either rotary (wheel) or nozzle atomizers.

Droplet sizes are typically in the range of from 10 to 100 micrometer depending on the atomization principle. There are two main types of nozzles: high pressure single fluid nozzle (50 to 500 bars) and two-fluid nozzles: one fluid is the liquid to dry and the second is a compressed gas (generally air at 2 to 7 bars).

Evaporation of the moisture from the droplets and the formation of dry particles take place under controlled temperature and air flow conditions. Powder is discharged continuously from the drying chamber. Operating conditions are selected according to the drying characteristics of the product of interest.

Any spray dryer known in the art may be used according to the present invention but in an embodiment of the method, the spray drying step is done using a Two-Fluid-Nozzle (TFN) or a Rotary Atomizer.

The spray-drying may be performed using an inlet temperature of from 100° C. to 200° C.; preferably an inlet temperature of from 120° C. to 200° C.; in particular an inlet temperature of from 140° C. to 200° C.

The spray-drying may be performed using an outlet temperature of from 40° C. to 95° C.; preferably an outlet temperature of from 50° C. to 94° C.; preferably an outlet temperature of from 60° C. to 94° C.; in particular an outlet temperature of from 70° C. to 93° C.

The spray-drying may be performed using a feed temperature of from 0° C. to 100° C.; e.g., a feed temperature of from 1° C. to 100° C.; a feed temperature of from 2° C. to 100° C.; a feed temperature of from 3° C. to 100° C.; a feed temperature of from 4° C. to 100° C.; a feed temperature of from 5° C. to 100° C.; a feed temperature of from 6° C. to 100° C.; a feed temperature of from 7° C. to 100° C.; a feed temperature of from 8° C. to 100° C.; a feed temperature of from 9° C. to 100° C.; a feed temperature of from 10° C. to 100° C.; a feed temperature of from 11° C. to 100° C.; a feed temperature of from 12° C. to 100° C.; a feed temperature of from 13° C. to 100° C.; a feed temperature of from 14° C. to 100° C.; a feed temperature of from 15° C. to 100° C.; a feed temperature of from 16° C. to 100° C.; a feed temperature of from 17° C. to 100° C.; a feed temperature of from 18° C. to 100° C.; a feed temperature of from 19° C. to 100° C.; a feed temperature of from 20° C. to 100° C.; a feed temperature of from 21° C. to 100° C.; a feed temperature of from 22° C. to 100° C.; a feed temperature of from 23° C. to 100° C.; a feed temperature of from 24° C. to 100° C.; a feed temperature of from 25° C. to 100° C.; a feed temperature of from 26° C. to 100° C.; a feed temperature of from 27° C. to 100° C.; a feed temperature of from 28° C. to 100° C.; a feed temperature of from 29° C. to 100° C.; a feed temperature of from 30° C. to 100° C.; a feed temperature of from 31° C. to 100° C.; a feed temperature of from 32° C. to 100° C.; a feed temperature of from 33° C. to 100° C.; a feed temperature of from 34° C. to 100° C.; a feed temperature of from 35° C. to 100° C.; a feed temperature of from 36° C. to 100° C.; a feed temperature of from 37° C. to 100° C.; a feed temperature of from 38° C. to 100° C.; a feed temperature of from 39° C. to 100° C.; a feed temperature of from 40° C. to 100° C.; a feed temperature of from 41° C. to 100° C.; a feed temperature of from 42° C. to 100° C.; a feed temperature of from 43° C. to 100° C.; a feed temperature of from 44° C. to 100° C.; a feed temperature of from 45° C. to 100° C.; a feed temperature of from 46° C. to 100° C.; a feed temperature of from 47° C. to 100° C.; a feed temperature of from 48° C. to 100° C.; a feed temperature of from 49° C. to 100° C.; a feed temperature of from 50° C. to 100° C.; a feed temperature of from 51° C. to 100° C.; a feed temperature of from 52° C. to 100° C.; a feed temperature of from 53° C. to 100° C.; a feed temperature of from 54° C. to 100° C.; a feed temperature of from 55° C. to 100° C.; a feed temperature of from 56° C. to 100° C.; a feed temperature of from 57° C. to 100° C.; a feed temperature of from 58° C. to 100° C.; a feed temperature of from 59° C. to 100° C.; a feed temperature of from 60° C. to 100° C.; a feed temperature of from 61° C. to 100° C.; a feed temperature of from 62° C. to 100° C.; a feed temperature of from 63° C. to 100° C.; a feed temperature of from 64° C. to 100° C.; a feed temperature of from 65° C. to 100° C.; a feed temperature of from 66° C. to 100° C.; a feed temperature of from 67° C. to 100° C.; a feed temperature of from 68° C. to 100° C.; a feed temperature of from 69° C. to 100° C.; a feed temperature of from 70° C. to 100° C.; in particular to a feed temperature of from 70° C. to 99° C.

The nozzle air temperature will typically be between 10° C. and 100° C.; in particular between 20° C. and 90° C.

The rotary atomizer peripheral speed will typically be between 50 m/s and 250 m/s.

It may be convenient first to produce the dry fine powder according to the invention by spray drying. Said fine powder may then be fluidized in a fluid bed and a liquid binder, e.g., water is sprayed into the equipment to build up the agglomerates.

### EXAMPLES

### Example 1

### Spray Drying Using Different Combinations of Hyaluronic Acid (HA) Concentration in Feed, Feed Temperature, and Atomization Principle

HA was run on Minor pilot scale spray dryer (MM) with different combinations of HA concentration, feed temperature and atomization principle (external TFN or rotary atomizer). All experiments were conducted on a GEA Mobile Minor pilot scale spray dryer (MM).

The following parameters were measured: HA MW in the feed to the spray dryer, HA MW in product, and particle size distribution (PSD).

All experiments were conducted with a drying chamber inlet temperature (Tᵢₙ) of 195° C., an outlet temperature (Tₒᵤₜ) of 85° C., and a drying air flow of around 80 kg/h.

Table 1 summarizes the results using Rotary as the atomization principle, and Table 2 summarizes the results using TFN as the atomization principle

TABLE 1

| | | | | | | | Aver. particle |
|---|---|---|---|---|---|---|---|
| Feed | HA conc. in | Atomization | HA MW in SD | HA MW in | MW loss, | MW | size, |
| temp, ° C. | feed, g/L | principle | product, MDa | feed, MDa | MDa | loss % | micrometer |
| 20 | 1 | Rotary | 1.16 | 1.30 | 0.14 | 10.8 | 7.2 |
| 95 | 1 | Rotary | 1.09 | 1.28 | 0.19 | 14.8 | 6.9 |
| 55 | 4 | Rotary | 1.29 | 1.30 | 0.01 | 0.8 | 10.5 |
| 55 | 4 | Rotary | 1.25 | 1.28 | 0.03 | 2.4 | 11.7 |
| 95 | 7 | Rotary | 1.24 | 1.31 | 0.07 | 5.3 | 16.2 |
| 18 | 7 | Rotary | 1.30 | 1.32 | 0.02 | 1.5 | 17.2 |

TABLE 2

| | | | | | | | Aver. particle |
|---|---|---|---|---|---|---|---|
| Feed | HA conc in | Atomization | HA MW in SD | HA MW in | MW loss, | MW | size, |
| temp, ° C. | feed, g/L | principle | product, MDa | feed, MDa | MDa | loss % | micrometer |
| 20 | 1 | External TFN | 0.998 | 1.30 | 0.302 | 23.2 | 3.9 |
| 95 | 1 | External TFN | 1.10 | 1.32 | 0.220 | 16.8 | 7.5 |
| 55 | 4 | External TFN | 1.19 | 1.31 | 0.120 | 9.1 | 6.3 |
| 95 | 7 | External TFN | 1.21 | 1.33 | 0.120 | 9.0 | 9.7 |
| 18 | 7 | External TFN | 1.23 | 1.30 | 0.070 | 5.3 | 7.7 |

None of the products showed any discoloration.

Statistical analysis of the results from Table 1 and Table 2 shows that using a high HA concentration in the feed results in a significant reduction in the MW loss during spray drying.

### Example 2

### Spray Drying of Hyaluronic Acid with Various Molecular Weights

Various batches of feed with hyaluronic acid (HA) of various HA molecular weights (MW) and concentrations were spray dried on a pilot scale fluidised spray dryer (FSD) equipped with an external two-fluid nozzle (TFN).

Table 3 gives an overview of concentration in the feed, HA molecular weights, and HA molecular weights of the dried product.

All batches were spray dried with drying chamber inlet temperature of 195° C., air outlet temperature of 89° C., and feed temperature of 95° C.

None of the products showed any discoloration.

TABLE 3

| | | | | | Aver. |
|---|---|---|---|---|---|
| HA conc. | HA MW in | HA MW | MW | MW | particle |
| in feed, | SD product, | in feed, | loss, | loss, | size, |
| g/L | MDa | MDa | MDa | % | micrometer |
| 1.8 | 1.22 | 1.79 | 0.570 | 31.8 | 4 |
| 3.3 | 1.01 | 1.23 | 0.220 | 17.9 | 6 |
| 3.7 | 1.08 | 1.21 | 0.130 | 10.7 | 6 |

### Example 3

### Spray Drying Using Different Combinations of Hyaluronic Acid (HA) Concentration in Feed, Feed Temperature, and Atomization Principle

HA was run on Minor pilot scale spray dryer (MM) with different combinations of HA concentration, feed temperature and atomization principle (external TFN or rotary atomizer). All experiments were conducted on a GEA Mobile Minor pilot scale spray dryer (MM).

The following parameters were measured: HA MW in feed, HA MW in product, and particle size distribution (PSD).

All experiments were conducted with a drying chamber inlet temperature (Tᵢₙ) of 195° C., an outlet temperature (Tₒᵤₜ) of 90° C., and a drying air flow of around 80 kg/h.

Table 4 summarizes the results:

TABLE 4

| | | | | | | | Aver. particle |
|---|---|---|---|---|---|---|---|
| Feed | HA conc. in | Atomization | HA MW in SD | HA MW in | MW loss, | MW | size, |
| temp, ° C. | feed, g/L | principle | product, MDa | feed, MDa | MDa | loss % | micrometer |
| 55 | 4 | Rotary | 1.29 | 1.30 | 0.010 | 0.77 | 11 |
| 55 | 4 | Rotary | 1.25 | 1.28 | 0.030 | 2.34 | 12 |
| 95 | 7 | Rotary | 1.24 | 1.31 | 0.070 | 5.34 | 16 |
| 95 | 7 | TFN | 1.21 | 1.33 | 0.120 | 9.02 | 10 |
| 18 | 7 | Rotary | 1.30 | 1.32 | 0.020 | 1.51 | 17 |
| 18 | 7 | External | 1.23 | 1.30 | 0.070 | 5.38 | 8 |
| 75 | 3 | Rotary | 1.24 | 1.28 | 0.040 | 3.13 | 9 |
| 87 | 4 | Rotary | 1.34 | 1.36 | 0.020 | 1.47 | 9 |
| 87 | 4 | Rotary | 1.30 | 1.34 | 0.040 | 2.99 | 6 |
| 87 | 4 | Rotary | 1.30 | 1.38 | 0.080 | 5.80 | 6 |
| 98 | 5 | Rotary | 1.31 | 1.35 | 0.040 | 2.96 | 15 |
| 75 | 5 | Rotary | 1.30 | 1.37 | 0.070 | 5.11 | 6 |
| 20 | 1 | Rotary | 1.45 | 1.82 | 0.37 | 20.4 | 6 |
| 20 | 1 | TFN | 1.21 | 1.84 | 0.63 | 34.2 | 5 |
| 95 | 1 | TFN | 1.27 | 1.86 | 0.59 | 31.7 | 6 |
| 95 | 1 | Rotary | 1.34 | 1.85 | 0.51 | 27.6 | 6 |
| 55 | 4 | Rotary | 1.80 | 1.90 | 0.10 | 5.26 | 7 |
| 55 | 4 | TFN | 1.53 | 1.91 | 0.38 | 19.9 | 7 |
| 95 | 7 | Rotary | 1.95 | 2.10 | 0.15 | 7.14 | n.d. |
| 95 | 7 | TFN | 1.54 | 1.99 | 0.45 | 22.6 | 10 |
| 20 | 7 | Rotary | 1.36 | 2.01 | 0.65 | 32.3 | 7 |
| 20 | 7 | TFN | 1.45 | 1.89 | 0.44 | 23.3 | 6 |

n.d.: not determined.

It can be seen from Table 4 that with a feed concentration of 1 g/l the molecular weight loss is high (20.4%; 34.2%; 31.7% and 27.6%).

It can be seen from Table 4 that a feed concentration of around 7 g/l seems to be the upper limit—some results are fine, and some results have a high molecular weight loss (22.6%; 32.3% and 23.3%).

1. A method of producing spray-dried hyaluronic acid comprising
a) spray drying hyaluronic acid, wherein the concentration of the hyaluronic acid in the feed to the spray dryer is in the range of from 3.5 g/l to 7.0 g/l;
b) having the temperature in the feed to the spray dryer in the range of from 0° C. to 100° C.; and
wherein the molecular weight of the hyaluronic acid in the feed to the spray dryer is ≧1200 kDa.

2. The method according to claim 1, wherein the hyaluronic acid is hyaluronic acid or a salt thereof.

3. The method according to claim 2, wherein the salt of the hyaluronic acid is selected from the group consisting of sodium hyaluronate, potassium hyaluronate, ammonium hyaluronate, calcium hyaluronate, magnesium hyaluronate, zinc hyaluronate, and cobalt hyaluronate.

4. The method according to claim 1, wherein the hyaluronic acid in the feed to the spray dryer has a molecular weight in the range of between 1200 kDa and 10,000 kDa.

5. The method according to claim 1, wherein the hyaluronic acid is derivatized.

6. The method according to claim 5, wherein the derivatized hyaluronic acid is aryl/alkyl succinic anhydride hyaluronic acid or acrylated hyaluronic acid.

7. The method according to claim 1, wherein the spray drying is done using a Rotary Atomizer or a Two-Fluid-Nozzle (TFN) spray-dryer.

8. The method according to claim 1, wherein the spray drying is done using the following conditions:
Inlet temperature: 100-200° C.; and
Outlet temperature: 40-95° C.

9. The method according to claim 1, wherein the loss in molecular weight during spray drying is less than 15%.

10. The method according to claim 1, wherein the feed to the spray dryer also comprises an active ingredient.

11. The method according to claim 1, wherein the feed to the spray dryer also comprises an excipient.
